SEQUENCE LISTING (1 sequence)

SEQ ID NO: 1 - DNA (other nucleic acid), 58 bp; single-stranded, linear
  description: /desc = “oligonucleotide”
aagcttatgaagcgcgcaagaccgtctgaagataccttcaaccccgtgtatccatatg

(19) United States
(12) Patent Application Publication
(10) Pub. No.: US 2003/0096787 A1
(43) Pub. Date: May 22, 2003

(54) Defective adenovirus vectors and use thereof in gene therapy

(76) Inventors: Michel Perricaudet, Ecrosnes (FR); Emmanuelle Vigne, Ivry-sur-Seine (FR); Patrice Yeh, Paris (FR)

Correspondence Address: BAKER & BOTTS, 30 ROCKEFELLER PLAZA, NEW YORK, NY, 10112

(21) Appl. No.: 10/301,085

(22) Filed: Nov. 21, 2002

Related U.S. Application Data
(63) Continuation of application No. 08/397,225, filed on Mar. 28, 1995.
Application No. 08/397,225 is a continuation of application No. PCT/FR94/00851, filed on Jul. 8, 1994.

(30) Foreign Application Priority Data
Jul. 13, 1993 (FR) 93/08596
Apr. 18, 1994 (FR) 94/04590

(51) Int. Cl. A61K 48/00

(52) U.S. Cl. 514/44; 424/93.2

(57) ABSTRACT

Novel adenovirus-derived viral vectors, the preparation thereof, and the use thereof in gene therapy, are disclosed.

The present invention relates to new viral vectors, their preparation and their use in gene therapy. It also relates to the pharmaceutical compositions containing the said viral vectors. More particularly, the present invention relates to recombinant adenoviruses as vectors for gene therapy.

Gene therapy consists in correcting a deficiency or an abnormality (mutation, aberrant expression and the like) by the introduction of a genetic information into the cell or affected organ. This genetic information can be introduced either in vitro or in a cell extracted from the organ, the modified cell then being reintroduced into the body, or directly in vivo into the appropriate tissue. In this second case, various techniques exist, among which various transfection techniques involving complexes of DNA and DEAE-dextran (Pagano et al., J. Virol. 1 (1967) 891), of DNA and nuclear proteins (Kaneda et al., Science 243 (1989) 375), of DNA and lipids (Felgner et al., PNAS 84 (1987) 7413), the use of liposomes (Fraley et al., J. Biol. Chem. 255 (1980) 10431) and the like. More recently, the use of viruses as vectors for the transfer of genes has appeared as a promising alternative to these physical transfection techniques. In this respect, various viruses have been tested for their capacity to infect certain cellular populations. In particular, the retroviruses (RSV, HMS, MMS and the like), the HSV virus, the adeno-associated viruses and the adenoviruses.

Among these viruses, adenoviruses present some advantageous properties for a use in gene therapy. Especially, they have a fairly broad host spectrum, are capable of infecting quiescent cells, do not integrate into the genome of the infected cell, and have not been associated to date with major pathologies in man.

Adenoviruses are viruses with linear double-stranded DNA of a size of about 36 kb. Their genome comprises especially an inverted repeat sequence (ITR) at their end, an encapsulation sequence, early genes and late genes (cf FIG. 1). The principal early genes are the E1 (E1a and E1b), E2, E3 and E4 genes. The principal late genes are the L1 to L5 genes.

Given the properties of the abovementioned adenoviruses, the latter have already been used for the transfer of genes in vivo. To this end, various vectors derived from adenoviruses have been prepared, incorporating various genes (β-gal, OTC, α-1AT, cytokines and the like). In each of these constructs, the adenovirus was modified so as to render it incapable of replication in the infected cell. Thus, the constructs described in the prior art are adenoviruses from which there have been deleted the E1 (E1a and/or E1b) and optionally E3 regions at the level of which the heterologous DNA sequences are inserted (Levrero et al., -Gene 101 (1991) 195; Gosh-Choudhury et al., Gene 50 (1986) 161). Nevertheless, the vectors described in the prior art have numerous disadvantages which limit their exploitation in gene therapy. In particular, all these vectors contain numerous viral genes whose expression in vivo is not desirable within the framework of a gene therapy. Furthermore, these vectors do not permit the incorporation of very large DNA fragments which may be necessary for certain applications.

The present invention makes it possible to overcome these disadvantages. The present invention indeed describes recombinant adenoviruses for gene therapy, which are capable of efficiently transferring DNA (up to 30 kb) in vivo, of expressing at high levels and in a stable manner this DNA in vivo, while limiting any risk of production of viral proteins, of transmission of the virus, of pathogenicity and the like. In particular, it was found that it is possible to considerably reduce the size of the adenovirus gene without preventing the formation of an encapsulated viral particle. This is surprising since it had been observed in the case of other viruses, for example retroviruses, that certain sequences distributed along the genome where necessary for an efficient encapsulation of the viral particles. Because of these, the production of vectors possessing substantial internal deletions was highly limited. The present invention also shows that neither does the suppression of most of the viral genes prevent the formation of such a viral particle. Furthermore, the recombinant adenoviruses thus obtained preserve, in spite of the substantial modifications of their genomic structure, their advantageous properties of high infectivity, of stability in vivo and the like.

The vectors of the invention are particularly advantageous since they permit the incorporation of desired DNA sequences of very large size. It is thus possible to insert a gene of a length greater than 30 kb. This is particularly advantageous for some pathologies whose treatment requires the co-expression of several genes, or the expression of very large genes. Thus, for example, in the case of muscular dystrophy, it was not until now possible to transfer the cDNA corresponding to the native gene responsible for this pathology (dystrophin gene) because of its large size (14 kb).

The vectors of the invention are also very advantageous since they possess very few functional viral regions and since, because of this, the risks inherent in the use of viruses as vectors in gene therapy such as immunogenicity, pathogenicity, transmission, application, recombination and the like, are substantially reduced or even suppressed.

The present-invention ‘thus provides viral ’ vectors which are particularly adapted to the transfer and expression in vivo of desired DNA sequences.

A first subject of the present invention therefore relates to a defective recombinant adenovirus comprising:

ITR sequences,

a sequence permitting the encapsulation,

a heterologous DNA sequence, and in which:

the E1 gene is non-functional and

at least one of the E2, E4 and L1-L5 genes is non-functional.

For the purposes of the present invention, the term “defective adenovirus” designates an adenovirus incapable of replicating autonomously in the target cell. Generally, the genome of the defective adenoviruses according to the present invention is therefore devoid of at least the sequences necessary for the replication of the said virus in the infected cell. These regions can be either removed (completely or partly), or rendered non-functional, or substituted by other sequences and especially by the heterologous DNA sequence.

The inverted repeat sequences (ITR) constitute the replication origin of the adenoviruses. They are localized at the 3′ and 5′ ends of the viral genome (cf FIG. 1), from where they can be easily isolated according to conventional molecular biology techniques known to persons skilled in the art. The nucleotide sequence of the ITR sequences of human adenoviruses (in particular of the Ad2 and Ad5 serotypes) is described in the literature, as well as of canine adenoviruses (especially CAV1 and CAV2). As regards the Ad5 adenovirus for example, the left ITR sequence corresponds to the region comprising nucleotides 1 to 103 of the genome.

The encapsulation sequence (also designated Psi sequence) is necessary for the encapsulation of the viral DNA. This region should therefore be present in order to permit the preparation of defective recombinant adenoviruses according to the invention. The encapsulation sequence is localized in the genome of adenoviruses, between the left (5′) ITR and the E1 gene (cf FIG. 1). It can be isolated or synthesized artificially by conventional molecular biology techniques. The nucleotide sequence of the encapsulation sequence of human adenoviruses (in particular of the Ad2 and Ad5 serotypes) is described in the literature, as well as of canine adenoviruses (especially CAV1 and CAV2). As regards the Ad5 adenovirus for example, the encapsulation sequence corresponds to the region comprising nucleotides 194 to 358 of the genome.

There are various adenovirus serotypes whose structure and properties vary somewhat. Nevertheless, these viruses exhibit a comparable genetic organization, and the information described in the present application can be easily reproduced by persons skilled in the art for any type of adenovirus.

The adenoviruses of the invention may be of human, animal or mixed (human and animal) origin.

As regards the adenoviruses of human origin, the use of those classified in group C is preferred. More preferably, among the various human adenovirus serotypes, the use of the type 2 or 5 adenoviruses (Ad2 or Ad5) is preferred within the framework of the present invention.

As indicated above, the adenoviruses of the invention may also be of animal origin, or contain sequences derived from adenoviruses of animal origin. The Applicant has indeed shown that the adenoviruses of animal origin are capable of infecting, with a high efficiency, human cells, and that they are incapable of propagating in the human cells in which they were tested (cf Application FR 93 05954). The Applicant also showed that the adenoviruses of animal origin are not at all transcomplemented by adenoviruses of human origin, which eliminates any risk of recombination and of propagation in vivo, in the presence of a human adenovirus, capable of leading to the formation of infectious particles. The use of adenoviruses or of adenovirus regions of animal origin is therefore particularly advantageous since the risks inherent in the use of viruses as vectors in gene therapy are even smaller.

The adenoviruses of animal origin which can be used within the framework of the present invention may be of canine, bovine, murine, (example: Mav1, Beard et al., Virology 75 (1990) 81), ovine, porcine or avian or alternatively simian origin (example: SAV). More particularly, among the avian adenoviruses, there may be mentioned the serotypes 1 to 10 which are available at ATCC, such as for example the strains Phelps (ATCC VR-432), Fontes (ATCC VR-280), P7-A (ATCC VR-827), IBH-2A (ATCC VR-828), J2-A (ATCC VR-829), T8-A (ATCC VR-830), K-11 (ATCC VR-921) or alternatively the strains referenced ATCC VR-831 to 835. Among the bovine adenoviruses, the various known serotypes can be used, and especially those available at ATCC (types 1 to 8) under the references ATCC VR-313, 314, 639-642, 768 and 769. There may also be mentioned the murine adenoviruses FL (ATCC VR-550) and E20308 (ATCC VR-528), the type 5 (ATCC VR-1343), or type 6 (ATCC VR-1340) ovine adenovirus; the porcine adenovirus 5359), or the simian adenoviruses such as especially the adenoviruses referenced at ATCC under the numbers VR-591-594, 941-943, 195-203 and the like.

Preferably, among the various adenoviruses of animal origin, adenoviruses or adenovirus regions of canine origin, and especially all the CAV2 adenovirus strains [manhattan or A26/61 strain (ATCC VR-800) for example] are used within the framework of the invention. The canine adenoviruses have been the subject of numerous structural studies. Thus, complete restriction maps of the CAV1 and CAV2 adenoviruses have been described in the prior art (Spibey et al., J. Gen. Virol, 70 (1989) 165), and the E1a and E3 genes as well as the ITR sequences have been cloned and sequenced (see especially Spibey et al., Virus Res. 14 (1989) 241; Linné, Virus Res. 23 (1992) 119, WO 91/11525).

As indicated above, the adenoviruses of the present invention contain a heterologous DNA sequence. The heterologous DNA sequence designates any DNA sequence introduced into the recombinant virus, whose transfer and/or expression in the target cell is desired.

In particular, the heterologous DNA sequence may contain one or more therapeutic genes and/or one or more genes encoding antigenic peptides.

The therapeutic genes which can thus be transferred are any gene whose transcription and optionally translation in the target cell generates products having a therapeutic effect.

This may be in particular genes encoding protein products having a therapeutic effect. The protein product thus encoded may be a protein, a peptide, an amino acid and the like. This protein product may be homologous with respect to the target cell (that is to say a product which is normally expressed in the target cell when the latter presents no pathology). In this case, the expression of a protein makes it possible for example to palliate an insufficient expression in the cell or the expression of an inactive or weakly active protein as a result of a modification, or alternatively to overexpress the said protein. The therapeutic gene may also encode a mutant of a cellular protein, having an increased stability, a modified activity and the like. The protein product may also be heterologous with respect to the target cell. In this case, an expressed protein can for example supplement or provide an activity deficient in the cell which enables it to combat a pathology.

Among the therapeutic products for the purposes of the present invention, there may be mentioned more particularly enzymes, blood derivatives, hormones, lymphokines: interleukins, interferons, TNF, and the like (FR 9203120), growth factors, neurotransmitters or their precursors or synthetic enzymes, trophic factors: BDNF, CNTF, NGF, IGF, GMF, aFGF, bFGF, NT3, NT5 and the like; apolipoproteins: ApoAI, ApoAIV, ApoE and the like (FR 93 05125), dystrophin or minidystrophin (FR 9111947), tumour suppressor genes: p53, Rb, Rap1A, DCC, k-rev and the like (FR 93 04745), the genes encoding factors involved in coagulation: Factors VII, VIII, IX and the like.

The therapeutic gene can also be an antisense gene or sequence, whose expression in the target cell makes it possible to control the expression of genes or the description of cellular mRNAs. Such sequences can for example be transcribed, in the target cell, into RNAs which are complementary to cellular mRNAs and thus block their translation into protein, according to the technique described in Patent EP 140 308.

As indicated above, the heterologous DNA sequence may also contain one or more genes encoding an antigenic peptide, capable of generating an immune response in man. In this particular implementational embodiment, the invention therefore permits the production of vaccines which make it possible to immunize man, especially against microorganisms or viruses. These may be especially antigenic peptides specific for the Epstein Barr virus, the HIV virus, the hepatitis B virus (EP 185 573), the pseudo-rabies virus, or alternatively specific for tumours (EP 259 212).

Generally, the heterologous DNA sequence also comprises sequences permitting the expression of the therapeutic gene and/or of the gene encoding the antigenic peptide in the infected cell. There may be sequences which are naturally responsible for the expression of the considered gene when these sequences are capable of functioning in the infected cell. They may also be sequences of different origin (responsible for the expression of other proteins, or even synthetic). In particular, they may be promotor sequences of eucaryotic or viral genes. For example, they may be promoter sequences derived from the genome of the cell which it is desired to infect. Likewise, they may be promotor sequences derived from the genome of a virus, including the adenovirus used. In this respect, there may be mentioned for example the of the E1A, MLP, CMV and RSV genes and the like. In addition, these expression sequences can be modified by addition of activating sequences, regulatory sequences and the like. Moreover, when the inserted gene does not contain expression sequences it can be inserted into the genome of the defective virus downstream of such a sequence.

Furthermore, the heterologus sequence may also contain, in particular upstream of the therapeutic gene a signal sequence directing the therapeutic product synthesized in the secretory pathways of the target cell. This signal sequence may be the natural signal sequence of the therapeutic product, but it may also be any other functional signal sequence, or an artificial signal sequence.

As indicated above, the vectors of the invention possess at least one of the non-functional E2, E4 and L1-L5 genes. The viral gene considered can be rendered non-functional by any technique known to a person skilled in the art, and especially by supression, substitution deletion or addition of one or more bases in the gene(s) considered. Such modifications can be obtained in vitro (on the isolated DNA) or in situ, for example, by means of genetic engineering techniques, or alternatively by treating with mutagenic agents.

Among the mutagenic agents, there may be mentioned for example physical agents such as energetic radiations (X-, g- and ultraviolet rays and the like), or chemical agents capable of reacting with various functional groups of the bases of the DNA, and for example alkylating agents [ethyl methanesulphonate (EMS), N-methyl-N′-nitro-N-nitrosoguanidine, N-nitroquinoline-1-oxide (NQO)], bialkylating agents, intercalating agents and the like.

By deletion, there is understood for the purposes of the invention, any suppression of the gene considered. This may be especially all or part of the coding region of the said gene, and/or all or part of the promotor region for transcription of the said gene. The suppression can be carried out by digestion by means of appropriate restriction enzymes, and then ligation, according to conventional molecular biology techniques, as illustrated in the examples.

The genetic modifications can also be obtained by gene disruption, for example according to the procedure initially described by Rothstein [Meth. Enzymol. 101 (1983) 2021. In this case, all or part of the coding sequence is preferably perturbed so as to permit the replacement, by homologous recombination, of the genomic sequence by a non-functional or mutant sequence.

The said genetic modification(s) may be localized in the coding part of the relevant gene, or outside the coding region, and for example in the regions responsible for the expression and/or transcriptional regulation of the said genes. The non-functional character of the said genes can therefore manifest itself by the production of an inactive protein because of structural or conformational modifications, by the absence of production, by the production of a protein having an altered activity, or alternatively by the production of the natural protein at an attenuated level or according to a desired mode of regulation.

Moreover, some alterations such as point mutations are, by nature, capable of being corrected or attenuated by cellular mechanisms. Such genetic alterations are then of a limited interest at the industrial level. It is therefore particularly preferred that the non-functional character is perfectly stable segregationally and/or non-reversible.

Preferably, the gene is non-functional because of a partial or total deletion.

Preferably, the defective recombinant adenoviruses of the invention are devoid of adenovirus late genes.

A particularly advantageous embodiment of the invention consists in a defective recombinant adenovirus comprising:

the ITR sequences,

a sequence permitting the encapsulation,

a heterologous DNA sequence, and

a region carrying the gene or a part of the gene E2.

Another particularly advantageous embodiment of the invention consists in a defective recombinant adenovirus comprising:

the ITR sequences,

a sequence permitting the encapsulation,

a heterologous DNA sequence, and

a region carrying the gene or a part of the gene E4.

Still in a particularly advantageous embodiment, the vectors of the invention possess, in addition, a functional gene E3 under the control of a heterologous ore preferably, the vectors possess part of the E3 gene permitting the expression of the protein gp19K.

The defective recombinant adenoviruses according to the invention can be prepared in various ways.

A first method consists in transfecting the DNA from the defective recombinant virus prepared in vitro (either by ligation, or in plasmid form) into a competent cell line, that is to say carrying in trans all the functions necessary for the complementation of the defective virus. These functions are preferably integrated in the genome of the cell, which makes it possible to avoid the risks of recombination, and confers increased stability on the cell line. The preparation of such cell lines is described in the examples.

A second approach consists in co-transfecting, into an appropriate cell line, the DNA from the defective recombinant virus prepared in vitro (either by ligation, or in plasmid form) and the DNA from a helper virus. According to this method, it is not necessary to have a competent cell line capable of complementing all the defective functions of the recombinant adenovirus. Part of these functions is indeed complemented by the helper virus. This helper virus should itself be defective and the cell line carries in trans the functions necessary for its complementation. The preparation of defective recombinant adenoviruses of the invention according to this method is also illustrated in the examples.

Among the cell lines which can be used within the framework of this second approach, there may be mentioned especially the human embryonic kidney line 293, the KB cells, the Hela, MDCK and GHK cells and the like (cf examples).

Then the vectors which have multiplied are recovered, purified and amplified according to conventional molecular biology techniques.

The present invention therefore also relates to the cell lines which can be infected by adenoviruses, comprising, integrated in their genome, the functions necessary for the complementation of a defective recombinant adenovirus as described above. In particular, it relates to the cell lines containing, integrated in their genome, the regions E1 and E2 (especially the region encoding the 72K protein) and/or E4 and/or the gene for the glucocorticoid receptor. Preferably, these lines are obtained from the 293 or gm DBP6 line.

The present invention also relates to any pharmaceutical composition comprising one or more defective recombinant adenoviruses as described above. The pharmaceutical compositions of the invention can be formulated for a topical, oral, parenteral, intranasal, intravenous, intramuscular, subcutaneous, intraocular and transdermal administration and the like.

Preferably, the pharmaceutical composition contains vehicles which are pharmaceutically acceptable for an injectable formulation. These may be in particular saline (monosodium or disodium phosphate, sodium, potassium, calcium or magnesium chloride and the like or mixtures of such salts), sterile or isotonic solutions, or dry, especially freeze-dried, compositions, which by addition, depending on the case, of sterilized water or physiological saline, permit the constitution of injectable solutions.

The virus doses used for the injection can be adapted as a function of various parameters, and especially as a function of the mode of administration used, the relevant pathology, the gene to be expressed, or alternatively the desired duration of the treatment. Generally, the recombinant adenoviruses according to the invention are formulated and administered in the form of doses of between 10⁴ and 10¹⁴ pfu/ml, and preferably 106 to 10¹⁰ pfu/ml. The term pfu (“plaque forming unit”) corresponds to the infectivity of a virus solution, and is determined by infecting an appropriate cell culture, and measuring, generally after 5 days, the number of plaques of infected cells. The techniques for the determination of the pfu titre of a viral solution are well documented in the literature.

Depending on the inserted heterologous DNA sequence, the adenoviruses of the invention can be used for the treatment or prevention of numerous pathologies including genetic diseases (dystrophy, cystic fibrosis and the like), neurogegenerative diseases (Alzheimer, Parkinson, ALS and the like), cancers, pathologies linked to coagulation disorders and to dyslipoproteinaemias, pathologies linked to viral infections (hepatitis, AIDS and the like) and the like.

The present invention will be more fully described with the aid of the following examples which should be considered as illustrative and non-limiting.

### LEGEND TO THE FIGURES

FIG. 1: Genetic organization of the Ad5 adenovirus. The complete sequence of Ad5 is available on database and enables persons skilled in the art to select or create any restriction site, and thus to isolate any region of the genome.

FIG. 2: Restriction map of the CAV2 adenovirus Manhattan strain (according to Spibey et al., previously cited).

FIG. 3: Construction of defective viruses of the invention by ligation.

FIG. 4: Construction of a recombinant virus carrying the E4 gene.

FIG. 5: Construction of a recombinant virus carrying the E2 gene.

FIG. 6: Construction and representation of the plasmid pPY32,

FIG. 7: Representation of the plasmid pPY55.

FIG. 8: Representation of the plasmid p2.

FIG. 9: Representation of the intermediate plasmid used for the construction of the plasmid pITRL5-E4.

FIG. 10: Representation of the plasmid pITRL5-E4.

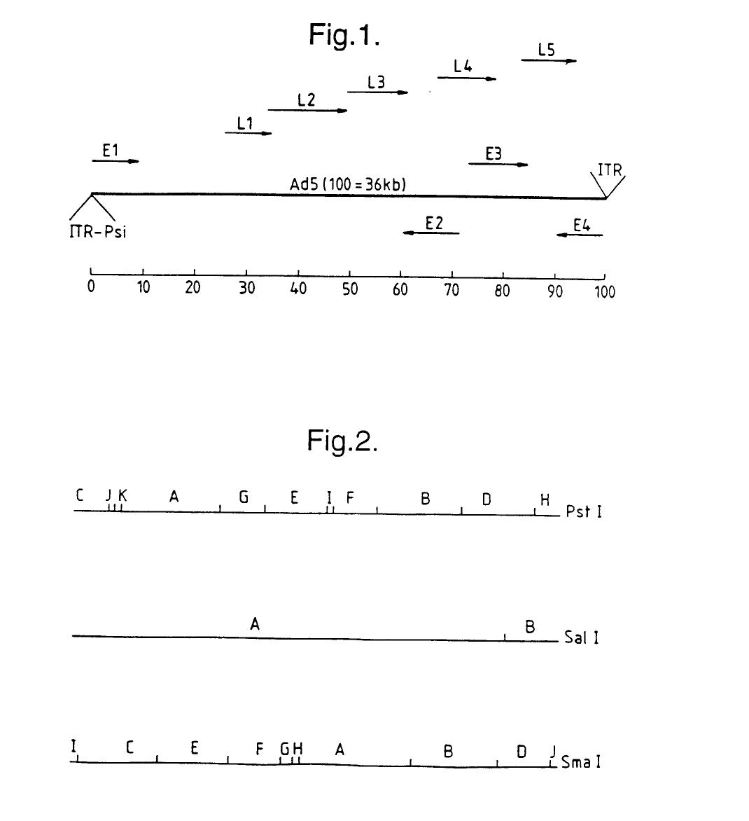

### GENERAL MOLECULAR BIOLOGY TECHNIQUES

The conventional methods used in molecular biology such as preparative extractions of plasmid DNA, centrifugation of plasmid DNA in cesium chloride gradient, electrophoresis on agarose or acrylamide gels, purification of DNA fragments by electroelution, protein extractions with phenol or phenol-chloroform, DNA precipitation in saline medium with ethanol or isopropanol, transformation in Escherichia coli and the like, are well known to persons skilled in the art and are widely described in the literature [Maniatis T. et al., “Molecular Cloning, a Laboratory Manual”, Cold Spring Harbor Laboratory, Cold Spring Harbor, N.Y., 1982; Ausubel F. M. et al. (eds), “Current Protocols in Molecular Biology”, John Wiley & Sons, New York, 1987].

The pBR322 and pUC type plasmids and the phages of the M13 series are of commercial origin (Bethesda Research Laboratories).

For the ligations, the DNA fragments can be separated according to their size by agarose or acrylamide gel electrophoresis, extracted with phenol or with a phenol/chloroform mixture, precipitated with ethanol and then incubated in the presence of phage T4 DNA ligase (Biolabs) according to the recommendations of the supplier.

The filling of the protruding 5′ ends can be performed with the Klenow fragment of DNA polymerase I of E. coli (Biolabs) according to the specifications of the supplier. The destruction of the protruding 3′ ends is performed in the presence of phage T4 DNA polymerase (Biolabs) which is used according to the recommendations of the manufacturer. The destruction of the protruding 5′ ends is performed by a controlled treatment with S1 nuclease.

The site-directed mutagenesis in vitro with synthetic oligodeoxynucleotides can be carried out according to the method developed by Taylor et al. [Nucleic Acids Res. 1 (1985) 8749-8764] using the kit distributed by Amersham.

The enzymatic amplification of DNA fragments by the so-called PCR technique Polymerase-catalyzed Chain Reaction, Saiki R. K. et al., Science 230 (1985) 1350-1354; Mullis K. B. and Faloona F. A., Meth. Enzym. 155 (1987) 335-350] can be carried out using the “DNA thermal cycler” (Perkin Elmer Cetus) according to the specifications of the manufacturer.

The verification of the nucleotide sequences can be carried out by the method developed by Sanger et al. [Proc. Natl. Acad. Sci. USA, 74 (1977) 5463-5467] using the kit distributed by Amersham.

Cell Lines Used

In the following examples, the following cell lines were or can be used:

Human embryonic kidney line 293 (Graham et al., J. Gen. Virol. 36 (1977) 59). This line contains especially, integrated in its genome, the left part of the genome of the human adenovirus Ad5 (12%).

Human cell line KB: derived from a human epidermal carcinoma, this line is available at ATCC (ref. CCL17) as well as the conditions permitting its culture.

Human cell line Hela: derived from a carcinoma of the human epithelium, this line is available at ATCC (ref. CCL2) as well as the conditions permitting its culture.

Canine cell line MDCK: the conditions for culture of the MDCK cells have been described especially by Macatney et al., Science 44 (1988)9.

Cell line gm DBP6 (Brough et al., Virology 190 (1992) 624). This line consists of Hela cells carrying the adenovirus E2 gene under the control of the LTR of MMTV.

### EXAMPLES

### Example 1

This example demonstrates the feasibility of a recombinant adenovirus devoid of most of the viral genes. For that, a series of adenovirus deletion mutants was constructed by ligation in vitro, and each of these mutants was co-transfected with a helper virus into the KB cells. These cells not permitting the propagation of the viruses defective for E1, the transcomplementation applies to the E1 region.

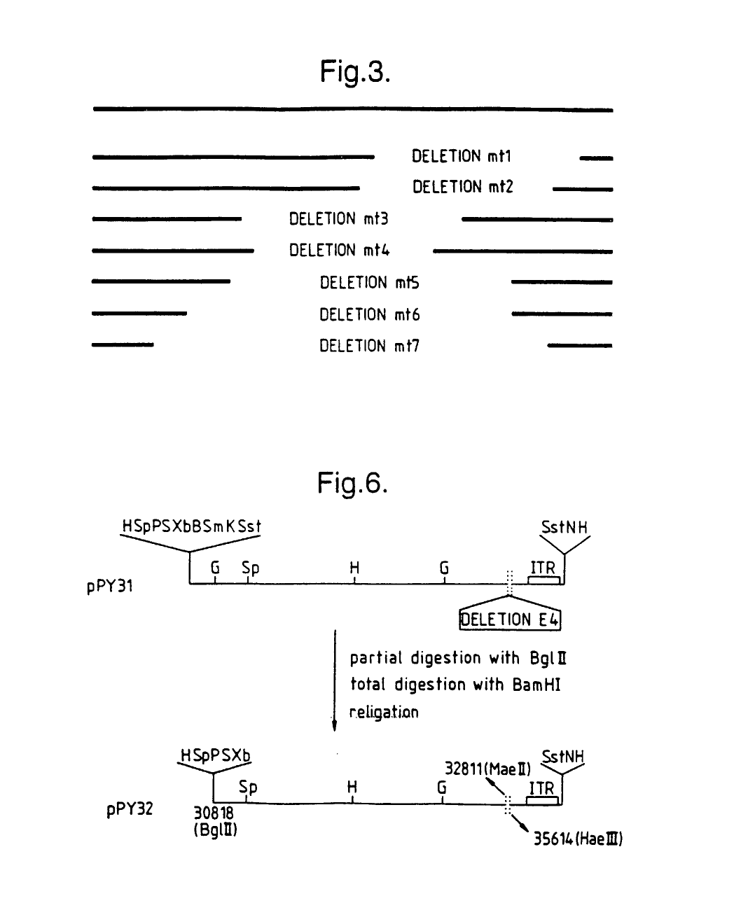

The various deletion mutants were prepared from the Ad5 adenovirus by digestion and then ligation in vitro. For that, the viral DNA from Ad5 is isolated according to the technique described by Lipp et al. (J. Virol. 63 (1989) 5133), subjected to digestion in the presence of various restriction enzymes (cf FIG. 3), and then the digestion product is ligated in the presence of T4 DNA ligase. The size of the various deletion mutants is then checked on a 0.8% SDS-agarose gel. These mutants are then mapped (cf FIG. 3). These various mutants contain the following regions:

mt1:Ligation between the Ad5 fragments 0-20642(SauI) and (SauI)33797-35935

mt2:Ligation between the Ad5 fragments 0-19549(NdeI) and (NdeI)31089-35935

mt3:Ligation between the Ad5 fragments 0-10754(AatII) and (AatII)25915-35935

mt4:Ligation between the Ad5 fragments 0-11311(MluI) and (MluI)24392-35935

mt5: Ligation between the Ad5 fragments 0-9462(SalI) and (XhoI)29791-35935

mt6: Ligation between the Ad5 fragments 0-5788(XhoI) and (XhoI)29791-35935

mt7: Ligation between the Ad5 fragments 0-3665(SphI) and (SphI)31224-35935

Each of the mutants prepared above was co-transfected with the viral DNA from Ad.RSVβGal (Stratford-Perricaudet et al., J. Clin. Invest. 90 (1992) 626) into the KB cells, in the presence of calcium phosphate. The cells were harvested 8 days after the transfection, and the culture supernatants were harvested and then amplified on KB cells until stocks of 50 dishes were obtained for each transfection. From each sample, episomal DNA was isolated and separated on cesium chloride gradient. Two distinct virus bands were observed in each case, collected and analysed. The heavier corresponds to the viral DNA from Ad.RSVβGal, and the lighter to the DNA from the recombinant virus generated by ligation (FIG. 3). The titre obtained for the latter is about 10⁸ pfu/ml.

A second series of adenovirus deletion mutants was constructed by ligation in vitro according to the same methodology. These various mutants contain the following regions:

mt8:Ligation between the fragments 0-4623(ApaI) from Ad RSVβGal and (ApaI)31909-35935 from Ad5.

mt9:Ligation between the fragments 0-10178(BglII) from Ad RSVβGal and (BamHI)21562-35935 from Ad5.

These mutants, containing the LacZ gene under the control of the LTR promotor of the RSV virus, are then co-transfected into the 293 cells in the presence of the viral DNA from H2d1808 (Weinberg et al., J. Virol. 57 (1986) 833), from which the E4 region is deleted. According to this second technique, the transcomplementation applies to E4 and no longer to E1. This technique thus makes it possible to generate, as described above, recombinant viruses possessing, as viral gene, only the E4 region.

### Example 2

This example describes the preparation of defective recombinant adenoviruses according to the invention by co-transfection, with a helper virus, of the DNA of the recombinant virus incorporated into a plasmid.

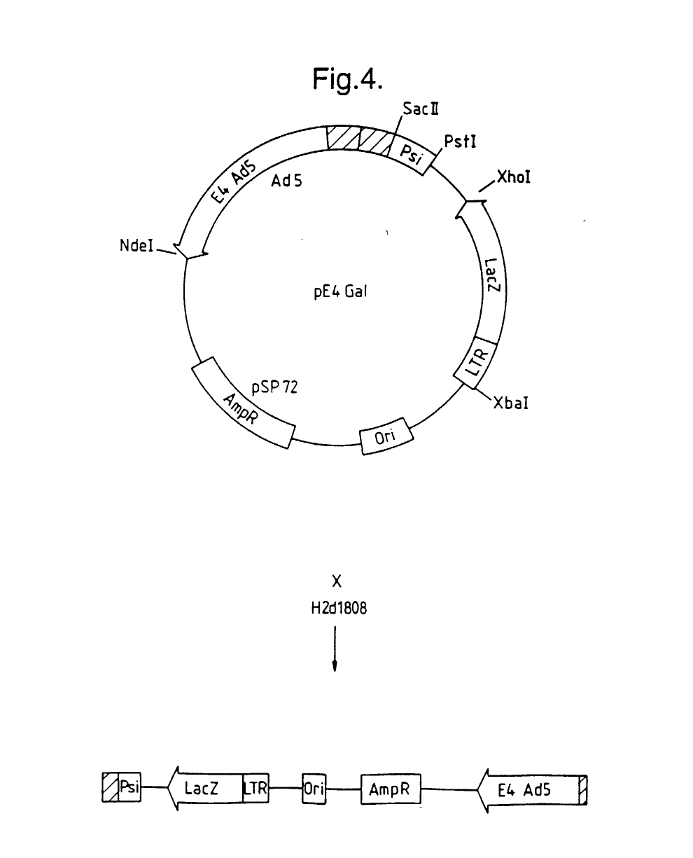

For that, a plasmid carrying the joining ITRs of Ad5, the encapsulation sequence, the E4 gene under the control of its own promotor and, as heterologous gene, the LacZ gene under the control of the LTR promotor of the RSV virus was constructed (FIG. 4). This plasmid, designated pE4Gal was obtained by cloning and ligation of the following fragments (see FIG. 4):

HindIII-SacII fragment derived from the plasmid pFG144 (Graham et al., EMBO J. 8 (1989) 2077). This fragment carries the ITR sequences from Ad5 in tandem and the encapsulation sequence: HindIII (34920)-SacII (352) fragment;

fragment from Ad5 between the SacII (localized at the level of the base pair 3827) and PstI (localized at the level of the base pair 4245) sites;

fragment of pSP 72 (Promega) between the PstI (bp 32) and SalI (bp 34) sites;

XhoI-XbaI fragment of the plasmid pAdLTR GalIX described in Stratford-Perricaudet et al. (JCI 90 (1992) 626). This fragment carries the LacZ gene under the control of the LTR of the RSV virus;

XbaI (bp 40)—NdeI (bp 2379) fragment of the plasmid pSP 72;

NdeI (bp 31089)—HindIII (bp 34930) fragment from Ad5. This fragment localized in the right end of the genome of Ad5, contains the E4 region under the control of its own promoter. It was cloned into the NdeI site (2379) of the plasmid pSP 72 and HindIII site of the first fragment.

This plasmid was obtained by cloning the various fragments into the indicated regions of the plasmid pSP 72. It is understood that equivalent fragments can be obtained by persons skilled in the art from other sources.

The plasmid pE4Gal is then co-transfected with the DNA from the virus H2d1808 into the 293 cells in the presence of calcium phosphate. The recombinant virus is then prepared as described in Example 1. This virus carries, as sole viral gene, the E4 gene from the Ad5 adenovirus (FIG. 4). Its genome has a size of about 12 kb, which permits the insertion of heterologous DNA of very large size (up to 20 kb). Thus, persons skilled in the art can easily replace the LacZ gene with any other therapeutic gene such as those mentioned above. Moveover, virus contains some sequences derived from the plasmid pSP 72, which can be removed by conventional molecular biology techniques if necessary.

### Example 3

This example describes the preparation of another defective recombinant adenovirus according to the invention by co-transfection, with a helper virus, of the DNA of the recombinant virus incorporated into a plasmid.

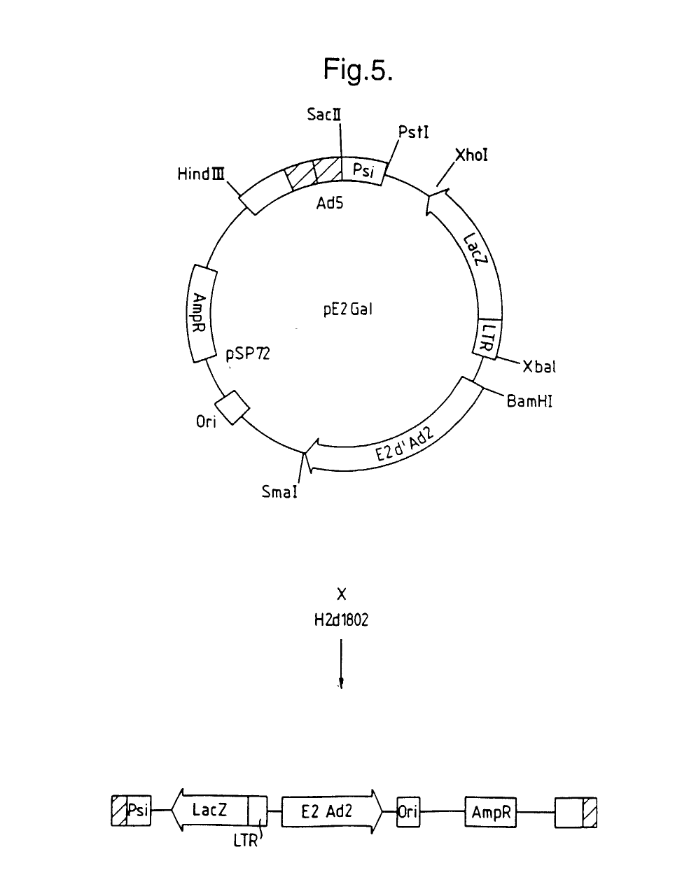

For that, a plasmid carrying the joining ITRs from Ad5, the encapsulation sequence, the E2 gene from Ad2 under the control of its own promoter and, as heterologous gene, the LacZ gene under the control of the LTR promoter of the RSV virus was constructed (FIG. 5). This plasmid, designated pE2Gal was obtained by cloning and ligation of the following fragments (see FIG. 5):

HindIII-SacII fragment derived from the plasmid pFG144 (Graham et al., EMBO J. 8 (1989) 2077). This fragment carries the ITR sequences from Ad5 in tandem and the encapsulation sequence: HindIII (34920)-SacII (352) fragment. It was cloned, with the following fragment into the HindIII (16)-PstI (32) sites of the plasmid pSP 72;

fragment from Ad5 between the SacII (localized at the level of the base pair 3827) and PstI (localized at the level of the base pair 4245) sites. This fragment was cloned into the SacII site of the preceding fragment and the PstI (32) site of the plasmid pSP 72;

fragment of pSP 72 (Promega) between the PstI (bp 32) and SalI (bp 34) sites;

XhoI-XbaI fragment of the plasmid pAdLTR GalIX described in Stratford-Perricaudet et al. (JCI 90(1992)626). This fragment carries the LacZ gene under the control of the LTR of the RSV virus. It was cloned into the SalI (34) and XbaI sites of the plasmid pSP 72.

fragment of pSP 72 (Promega) between the XbaI (bp 34) and BamHI (bp 46) sites;

BamHI (bp 21606)—SmaI (bp 27339) fragment of Ad2. This fragment of the Ad2 genome contains the E2 region under the control of its own promoter. It was cloned into the BamHI(46) and EcoRV sites of the plasmid pSP 72;

EcoRV(bp 81)—HindIII(bp 16) fragment of the plasmid pSP 72.

This plasmid was obtained by cloning the various fragments into the indicated regions of the plasmid pSP 72. It is understood that equivalent fragments can be obtained by persons skilled in the art from other sources.

The plasmid pE2Gal is then co-transfected with the DNA from the H2d1802 virus devoid of the E2 region (Rice et al. J. Virol. 56(1985)767) into the 293 cells, in the presence of calcium phosphate. The recombinant virus is then prepared as described in Example 1. This virus carries, as sole viral gene, the E2 gene from the Ad2 adenovirus (FIG. 5). Its genome has a size of about 12 kb, which permits the insertion of heterologous DNA of very large size (up to 20 kb). Thus, persons skilled in the art can easily replace the LacZ gene with any other therapeutic gene such as those mentioned above. Moreover, this virus contains some sequences derived from the intermediate plasmid, which can be removed by conventional molecular biology techniques if necessary.

### Example 4

This example describes the construction of complementing cell lines for the E1, E2 and/or E4 regions of adenoviruses. These lines permit the construction of recombinant adenoviruses according to the invention deleted for these regions, without having recourse to a helper virus. These viruses are obtained by in vivo recombination, and may contain major heterlogous sequences.

In the cell lines described, the E2 and E4 regions, which are potentially cytotoxic, are placed under the control of an inducible promoter: the LTR of MMTV (Pharmacia) which is induced by dexamethasone. It is understood that other promoters can be used, and especially LTR variants from MMTV carrying for example heterologous regulatory regions (especially “enhancer” region). The lines of the invention were constructed by transfecting the corresponding cells, in the presence of calcium phosphate, with a DNA fragment carrying the indicated genes (adenovirus regions and/or the gene for the glucocorticoid receptor) under the control of a transcription promoter and a terminator (polyadenylation site). The terminator may be either the natural terminator of the transfected gene, or a different terminator such as for example the terminator of the early messenger of the SV40 virus. Advantageously, the DNA fragment also carries a gene permitting the selection of the transformed cells, and for example, the gene for resistance to geneticin. The resistance gene can also be carried by a different DNA fragment, co-transfected with the first.

After transfection, the transformed cells are selected and their DNA is analysed in order to verify the integration of the DNA fragment into the genome.

This technique makes it possible to obtain the following cell lines:

1. 293 cells possessing the 72K gene of the E2 region of Ad5 under the control of the LTR of MMTV;

2. 293 cells possessing the gene for the 72K of the E2 region of Ad5 under the control of the LTR of MMTV and the gene for the glucocorticoid receptor;

3. 293 cells possessing the 72K gene of the E2 region of Ad5 under the control of the LTR of MMTV and the E4 region under the control of the LTR of MMTV;

4. 293 cells possessing the 72K gene of the E2 region of Ad5 under the control of the LTR of MMTV, the E4 region under the control of the LTR of MMTV and the gene for the glucocorticoid receptor;

5. 293 cells possessing the E4 region under the control of the LTR of MMTV;

6. 293 cells possessing the E4 region under the control of the LTR of MMTV and the gene for the glucorticoid receptor;

7. gm DBP6 cells possessing the E1A and E1B regions under the control of their own promoter;

8. gm DBP6 cells possessing the E1A and E1B regions under the control of their own promoter and the E4 region under the control of the LTR of MMTV.

### Example 5

This example describes the preparation of defective recombinant adenoviruses according to the invention from whose genome the E1, E3 and E4 genes are deleted. According to an advantageous embodiment, illustrated in this example and in Example 3 in particular, the genome of the recombinant adenoviruses of the invention is modified so that at least the E1 and E4 genes are non-functional. Such adenoviruses possess, first of all, a large capacity to incorporate heterologous genes. Moreover, these vectors are highly safe because of the deletion of the E4 region, which is involved in the regulation of the expression of the late genes, in the stability of the late nuclear RNAs, in the extinction of the expression of the proteins of the host cell and in the efficiency of the replication of the viral DNA. These vectors therefore possess a transcriptional background noise and a viral gene expression which are highly reduced. Finally, in a particularly advantageous manner, these vectors can be produced at titres comparable with the wild-type adenoviruses.

These adenoviruses were prepared from the plasmid pPY55, carrying the modified right part of the genome of the Ad5 adenovirus, either by co-transfection with a helper plasmid (also see Examples 1, 2 and 3), or by means of a complementing line (Example 4).

5.1 Construction of the Plasmid pPY55

a) Construction of the Plasmid pPY32

The AvrII-BcII fragment of the plasmid pFG144[F. L. Graham et al. EMBO J. 8(1989) 2077-2085], corresponding to the right end of the genome of the Ad5 adenovirus, was first cloned between the XbaI and BamHI sites of the vector pICl9H, prepared from a dam-context. This generates the plasmid pPY23. One advantageous characteristic of the plasmid pPY23 is that the SalI site obtained from the multiple cloning site of the vector pIC19H remains unique and that it is localized beside the right end of the genome of the Ad5 adenovirus. The HaeIII-SalI fragment of the plasmid pPY23 which contains the right end of the genome of the Ad5 adenovirus, from the HaeIII site localized in position 35614, was then cloned between the EcoRV and XhoI sites of the vector pIC20H, which generates the plasmid pPY29. One advantageous characteristic of this plasmid is that the XbaI and ClaI sites obtained from the multiple cloning site of the vector pIC20H are localized besides the EcoRV/HaeIII junction resulting from the cloning. Furthermore, this junction modifies the nucleotide context immediately adjacent to the ClaI site which has now become methylable in a dam+context. The XbaI(30470)-MaeII(32811) fragment of the genome of the Ad5 adenovirus was then cloned between the XbaI and ClaI sites of the plasmid pPY29 prepared from a dam-context, which generates the plasmid pPY30. The SstI fragment of the plasmid pPY30, which corresponds to the sequence of the genome of the Ad5 adenovirus from the SstI site in position 30556 up to the right end was finally cloned between the SstI sites of the vector pIC20H, which generates the plasmid pPY31, of which a restriction map of the insert localized between the HindIII sites is given in FIG. 6.

The plasmid pPY32 was obtained after partial digestion of the plasmid pPY31 with BglII followed by a total digestion with BamHI, and then religation. The plasmid pPY32 therefore corresponds to the deletion of the genome of the Ad5 adenovirus situated between the BamHI site of the plasmid pPY31 and the BglII site localized in position 30818. A restriction map of the HindIII fragment of the plasmid pPY32 is given in FIG. 6. One characteristic of the plasmid pPY32 is that it possesses unique SalI and XbaI sites.

b) Construction of the Plasmid pPY47

The BamHI(21562)-XbaI(28592) fragment of the genome of the Ad5 adenovirus was first cloned between the BamHI and XbaI sites of the vector p1C19H prepared from a dam-context, which generates the plasmid pPY17. This plasmid therefore contains a HindIII (26328)-BglII(28133) fragment of the genome of the Ad5 adenovirus, which can be cloned between the HindIII and BglII sites of the vector pIC20R, to generate the plasmid pPY34. One characteristic of this plasmid is that the BamHI site obtained from the multiple cloning site is localized within the immediate vicinity of the HindIII(26328) site of the genome of the Ad5 adenovirus.

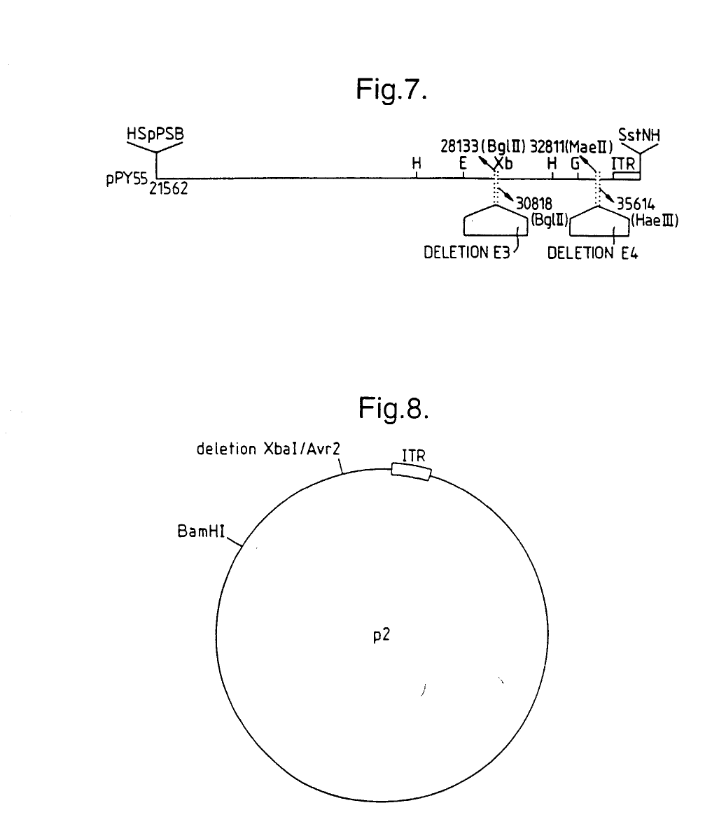

The BamHI21562)-HindIII(26328) fragment of the genome of the Ad5 adenovirus obtained from the plasmid pPY17 was then cloned between the BamHI and HindIII sites of the plasmid pPY34 which generates the plasmid pPY39. The BamHI-XbaI fragment of the plasmid pPY39 prepared from a dam-context, containing the part of the genome of the Ad5 adenovirus between the BamHI(21562) and BglII(28133) sites, was then cloned between the BamHI and XbaI sites of the vector pICl9H prepared from a dam-context. This generates the plasmid pPY47 of which one advantageous characteristic is that the SalI site obtained from the multiple cloning site is localized within the vicinity of the HindIII site (FIG. 7).

c) Construction of the Plasmid pPY55

The SalI-XbaI fragment of the plasmid pPY47 prepared from a dam-context, and which contains the part of the genome of the Ad5 adenovirus stretching from the BamHI(21562) site up to the BglII(28133) site, was cloned between the SalI and XbaI sites of the plasmid pPY32, which generates the plasmid pPY55. This plasmid can be directly used to produce recombinant adenoviruses which are at least deleted for the E3 region (deletion between the BglII sites localized at positions 28133 and 30818 of the genome of the Ad5 adenovirus) and for the entire E4 region (deletion between the MaeII (32811) and HaeIII (35614) sites of the genome of the Ad5 adenovirus (FIG. 7).

5.2 Preparation of the adenoviruses comprising at least one deletion in the E4 region, and preferably at least in the E1 and E4 regions.

a) Preparation by Co-Transfection with a Helper Virus E4 into the 293 Cells

The principle is based on the transcomplementation between a “mini-virus” (helper virus) expressing the E4 region and a recombinant virus deleted at least for E3 and E4. These viruses are obtained either by ligation in vitro, or after recombination in vivo, according to the following strategies:

(i) The DNA from the Ad-d1324 virus (Thimmappaya et al., Cell 31 (1982) 543) and the plasmid pPY55, both digested with BamHI, are first ligated in vitro, and then co-transfected with the plasmid pEAGal (described in Example 2) into the 293 cells.

(ii) The DNA from the Ad-d1324 virus digested with EcoRI and the plasmid pPY55 digested with BamHI are co-transfected, with the plasmid pE4Gal, into the 293 cells.

(iii) The DNA from the Ad5 adenovirus and the plasmid pPY55, both digested with BamHI are ligated and then co-transfected with the plasmid pE4Gal into the 293 cells.

(iv) The DNA from the Ad5 adenovirus digested with EcoRI and the plasmid pPY55 digested with BamHI are co-transfected with pEAGal into the 293 cells.

The strategies (i) and (ii) make it possible to generate a recombinant adenovirus deleted for the E1, E3 and E4 regions; the strategies (iii) and (iv) make it possible to generate a recombinant adenovirus deleted for the E3 and E4 regions. Of course, the DNA from a recombinant virus deleted for the E1 region but expressing any transgene can be used in place of the DNA from the Ad-d1324 virus according to strategies (i) or (ii), with the aim of generating a recombinant virus deleted for the E1, E3 and E4 regions and expressing the said transgene.

b) Preparation by Means of Cell Lines Transcomplementing the E1 and E4 Functions

The principle is based here on the fact that a cell line derived from a line expressing the E1 region, for example the line 293, and also expressing at least the open frames ORF6 and ORF6/7 of the E4 region of the Ad5 adenovirus under the control of a promoter, which is for example inducible, is capable of transcomplementing both for the E1 and E4 regions of the Ad5 adenovirus. Such lines were described in Example 4.

A recombinant virus deleted for the E1, E3 and E4 regions can therefore be obtained by ligation in vitro or recombination in vivo according to the procedures describe above. Regardless of the procedure used for generating the viruses deleted at least for the E4 region, a cytopathic effect (indicating the production of recombinant viruses) was observed after transfection into the cells used. The cells were then harvested, disrupted by three freeze-thaw cycles in their supernatant, and then centrifuged at 4000 rpm for 10 minutes. The supernatant thus obtained was then amplified on a fresh cell culture (293 cells for the procedures a) and 293 cells expressing the E4 region for the protocol b)). The viruses were then purified from the plaques and their DNA is analysed according to the method of Hirt (previously cited). The virus stocks are then prepared on cesium chloride gradient.

### Example 6

This example describes the preparation of defective recombinant adenoviruses according to the invention from whose genome the E1, E3 L5 and E4 genes are deleted. These vectors are particularly advantageous since the L5 region encodes the fiber, which is an extremely toxic protein for the cell.

These adenoviruses were prepared from the plasmid p2, carrying the modified right part of the genome of the Ad5 adenovirus, by co-transfection with various helper plasmids. They can also be prepared by means of a complementing line.

6.1 Construction of Plasmid p2

This plasmid contains all the right region of the genome of the Ad5 adenovirus, from the BamHI(21562) site, from which the fragment between the XbaI(28592) and AvrII(35463) sites, carrying the E3, L5 and E4 genes has been deleted. The plasmid p2 was obtained by cloning and ligating the following fragments into the plasmid pIC19R linearized with BamHI and dephosphorylated (see FIG. 8):

fragment of the genome of the Ad5 adenovirus between the BamHI(21562) and XbaI(28592) sites, and

right end of the genome of the Ad5 adenovirus (containing the right ITR), from the AvrII(35463) site up to the BclI site (BamHI compatible).

6.2. Construction of a Helper Plasmid (pITRL5-E4) Carrying the L5 Gene

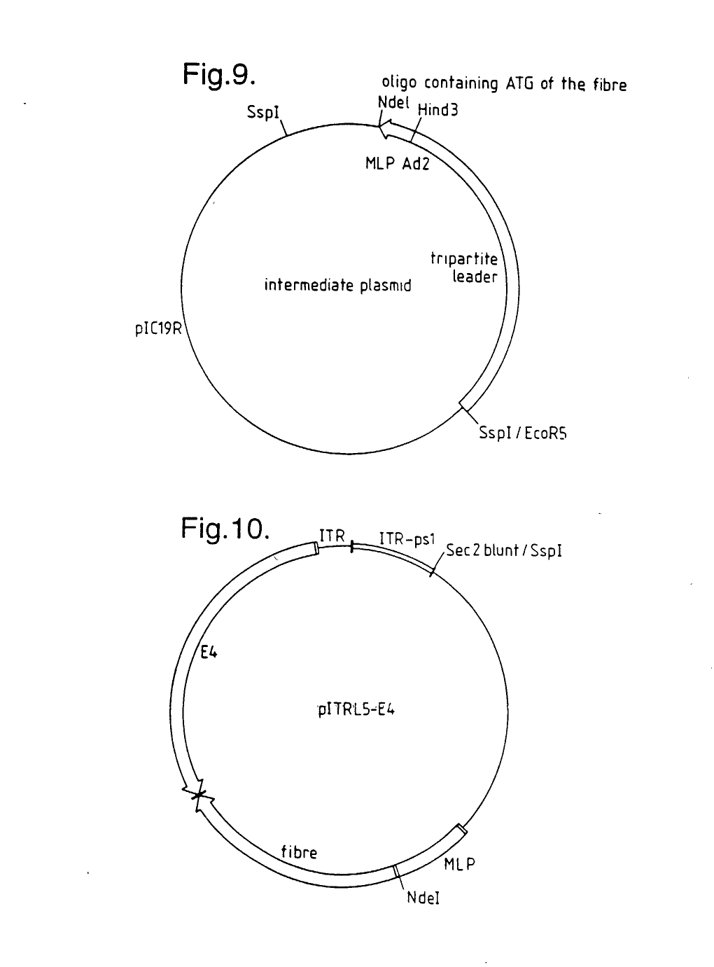

The helper plasmid pITRL5-E4 provides in trans the E4 and L5 genes. It corresponds to the plasmid pE4Gal described in Example 2, containing, in addition, the L5 region encoding the fiber under the control of the MLP promoter of the Ad2 adenovirus. The plasmid pITRL5-E4 was constructed in the following manner (FIGS. 9 and 10):

A 58 bp oligonucletide containing in the 5′-3′ direction, a HindIII site, the ATG of the fiber and the coding sequence of the fiber up to the NdeI site in position 31089 of the genome of the Ad5 adenovirus was synthesized. The sequence of this oligonucleotide is given below, in the 5′-3′ orientation: AAGCTTATGAAGCGCGCAAGACCGTCTGAAGATACCTTCAACCCCGTGTATCCAT ATG The HindIII site in 5′ and NdeI site in 3′, are underlined with a single line, the ATG of the fiber is underlined with a double line.

A SspI-HindIII fragment containing the sequence of the MLP promoter followed by the tripartite leader of the Ad2 adenovirus was isolated from the plasmid PMLP10 (Ballay et al.,(1987) UCLA Symposia on molecular and cellular biology, New series, Vol 70, Robinson et al (Eds) New-York, 481). This fragment was inserted with the 58 bp oligonucleotide described above between the NdeI and EcoRV sites of the plasmid pIC19R, to give an intermediate plasmid (see FIG. 9). The SacII (rendered blunt)-NdeI fragment of the plasmid pE4Gal (Example 2) was then introduced into the intermediate plasmid between the SspI and NdeI sites in order to generate the plasmid pITRL5-E4 (FIG. 10).

6.3 Preparation of the Defective Recombinant adenoviruses comprising a deletion in the E1, E3, L5 and E4 regions.

a) Preparation by Co-Transfection with a Helper Virus Into the 293 Cells.

The principle is based on the transcomplementation between a “mini-virus” (helper virus) expressing the L5 region or the E4 and L5 regions and a recombinant virus deleted at least for E3, E4 and L5.

These viruses were obtained either by ligation in vitro or after recombination in vivo, according to the following strategies:

(i) The DNA from the Ad-d1324 virus (Thimmappaya et al., Cell 31 (1982) 543) and the plasmid p2, both digested with BamHI, were first ligated in vitro and then co-transfected with the helper plasmid pITRL5-E4 (Example 6.2.) into the 293 cells.

(ii) The DNA from the Ad-d1324 virus digested with EcoRI and the plasmid p2 digested with BamHI are co-transfected with the plasmid pITRL5-E4 into the 293 cells.

(iii) The DNA from the Ad5 adenovirus and the plasmid p2, both digested with BamHI, are ligated and then co-transfected with the plasmid pITRL5-E4 into the 293 cells.

(iv) The DNA from the Ad5 adenovirus digested with EcoRI and the plasmid p2 digested with BamHI are co-transfected with pITRL5-E4 into the 293 cells.

The strategies (i) and (ii) make it possible to generate a recombinant adenovirus deleted for the E1 E3, L5 and E4 regions; the strategies (iii) and (iv) make it possible to generate a recombinant adenovirus deleted for the E3, L5 and E4 regions, of course, the DNA from a recombinant virus deleted for the E1 region but expressing any transgene can be used in place of the DNA from the Ad-d1324 virus according to strategies (i) or (ii), with the aim of generating a recombinant virus deleted for the E1, E3, L5 and E4 regions and expressing the said transgene.

The procedures described above can also be used with a helper virus carrying only the E5 region, using a cell line capable of expressing the E1 and E4 regions of the adenovirus, as described in Example 4.

Moreover, it is also possible to use a complementing line capable of expressing the E1, E4 and L5 regions, so as to completely avoid the use of a helper virus.

After the transfection, the viruses produced are recovered, amplified and purified under the conditions described in Example 5.

1. A defective recombinant adenovirus comprising
the ITR sequences,
a sequence permitting the encapsulation,
a heterologous DNA sequence,
and in which the E1 gene and at least one of the E2, E4 and L1-L5 genes is non-functional.

2. An adenovirus according to claim 1, characterized in that it is of human, animal or mixed origin.

3. An adenovirus according to claim 2, characterized in that the adenoviruses of human origin are chosen from those classified in group C, preferably from the type 2 or 5 adenoviruses (Ad2 or Ad5).

4. An adenovirus according to claim 2, characterized in that the adenoviruses of animal origin are chosen from adenoviruses of canine, murine, ovine, porcine, avian or simian origin.

5. An adenovirus according to one of the preceding claims, characterized in that at least the E1 and E4 genes are non-functional.

6. An adenovirus according to one of the preceding claims, characterized in that it is devoid of late genes.

7. An adenovirus according to claim 1, characterized in that it comprises;
the ITR sequence
a sequence permitting the encapsulation,
a heterologous DNA sequence, and
a region car the gene or part of the gene E2.

8. An adenovirus according to claim 1, characterized in that it comprises:
the ITR sequence
a sequence permitting the encapsulation,
a heterologous DNA sequence, and
a region carrying the gene or part of the gene E4.

9. An adenovirus according to claim 1, characterized that the E1, E3 and E4 genes are deleted from its genome.

10. An adenovirus according to claim 1, characterized in that the E1, L5 and E4 genes are deleted from it genome.

11. An adenovirus according to one of the preceding claims, characterized in that it comprises, in addition, a functional gene E3 under the control of a heterologous promoter.

12. An adenovirus according to one of the preceding claims, characterized in that the heterologous DNA sequence contains one or more therapeutic genes and/or one or more genes encoding antigenic peptides.

13. An adenovirus according to claim 12, characterized in that the therapeutic gene is chosen from the genes encoding enzymes, blood derivatives, hormones, lymphokines (interleukins, interferons, TNF and the like), growth factors, neurotransmitters or their precursors or synthetic enzymes, trophic factors (EDNF, NTF, NGF, IGF, GMF, aFGF, bFGF, NT3, NT5 and the like), apolipoproteins (ApoAI, ApoAIV, ApoE and the like), dystrophin or a minidystrophin, tumor suppressor genes or genes encoding factors involved in coagulation (Factors VII, VIII, and the like).

14. An adenovirus according to claim 12, characterized in that the therapeutic gene is an antisense gene or sequence whose expression in the target cell makes it possible to control the expression of genes or the transcription of cellular mRNAs.

15. An adenovirus according to claim 12, characterized in that the gene encodes an antigenic peptide capable of generating an immune response in man against microorganisms or viruses.

16. An adenovirus according to claim 15, characterized in that the gene encodes an antigenic peptide specific for the Epstein Barr virus, the HIV virus, the hepatitis B virus, the pseudo-rabies virus or alternatively specific for tumours.

17. An adenovirus according to one of the preceding claims, characterized in that the heterologous DNA sequence also comprises sequences permitting the expression of the therapeutic gene and/or of the gene encoding the antigenic peptide in the infected cell.

18. An adenovirus according to one of the preceding claims, characterized in that the heterologous DNA sequence comprises, upstream of the therapeutic gene, a signal sequence directing the therapeutic product synthesized in the secretory pathways of the target cell.

19. A cell line infectible by an adenovirus comprising, integrated into its genome, the functions necessary for the complementation of a defective recombinant adenovirus according to one of claims 1 to 18.

20. A cell line according to claim 19, characterized in that it contains, in its genome, at least the E1 and E2 genes from an adenovirus.

21. A cell line according to claim 20, characterized in that it contains, in addition, the E4 gene from an adenovirus.

22. A cell line according to claim 19, characterized in that it contains, in its genome, at least the E1 and E4 genes from an adenovirus.

23. A cell line according to claims 19 to 22, characterized in that it contains, in addition, the gene for the glucocorticold receptor.

24. A cell line according to claims 19 to 23, characterized in that the E2 and E4 genes are placed under the control of an inducible promoter.

25. A cell line according to claim 24, characterized in that the inducible promoter is the LTR promoter of MMTV.

26. A cell line according to claims 19 to 25 characterized in that the E2gene encodes the 72 K protein.

27. A cell line according to claims 19 to 26, characterized in that it is obtained from the line 293.

28. A pharmaceutical composition comprising at least one defective recombinant adenovirus according to one of claims 1 to 18.

29. A pharmaceutical composition according to claim 28, comprising a recombinant adenovirus according to one of claims 5 to 10.

30. A pharmaceutical composition according to claims 28 or 29, comprising a vehicle pharmaceutically acceptable for an injectable formulation.
